(19) United States
(12) Patent Application Publication
(10) Pub. No.: US 2024/0325591 A1
(43) Pub. Date: Oct. 3, 2024

(54) FILMS WITH MALODOR CONTROL

(71) Applicant: Berry Global, Inc., Evansville, IN (US)

(72) Inventors: Vipul Suhas Padman, Loveland, OH (US); Anna Renee Haney, Cincinnati, OH (US); Linda Lin, Mason, OH (US)

(21) Appl. No.: 18/593,104

(22) Filed: Mar. 1, 2024

Related U.S. Application Data
(60) Provisional application No. 63/449,760, filed on Mar. 3, 2023.

(51) Int. Cl. A61L 15/46; A61L 15/42; B29C 48/00; B29C 48/08; B29D 7/01; B29K 23/00; B29K 105/04; B29K 105/16

(52) U.S. Cl. CPC A61L 15/46; A61L 15/425; B29C 48/0018; B29C 48/022; B29C 48/08; B29D 7/01; B29K 2023/06; B29K 2023/12; B29K 2105/041; B29K 2105/16; B29K 2995/0065; B29K 2995/0069

(57) ABSTRACT

A breathable film comprising a vapor-permeable and liquid impermeable (VPLI) film comprising a microporous film including a plurality of micropores are provided. The breathable films include (a) a first outermost surface, (b) a second outermost surface, (c) a thickness extending between the first outermost surface and the second outermost surface, and (d) at least one odorous compound sequestering agent (OCSA) dispersed throughout the thickness of the breathable film. The at least one OCSA comprises (i) one or more salts of ricinoleic acid, and/or (ii) one or more cucurbituril compounds, such as CB[5], CB[6], CB[7], CB[8], or any mixture thereof, and/or (iii) one or more zeolites, and/or (iv) one or more halo active aromatic sulfonamide compounds. Absorbent articles including such breathable films are also provided.

### CROSS-REFERENCE TO RELATED APPLICATIONS

This application claims priority under 35 U.S.C. § 119 to U.S. Patent Application No. 63/449,760 filed Mar. 3, 2023, which is expressly incorporated by reference herein in its entirety.

### TECHNICAL FIELD

Embodiments of the presently-disclosed invention relate generally to films, such as breathable microporous films or non-breathable films, having at least one odorous compound sequestering agent (OCSA) applied topically and/or dispersed throughout the thickness of the film, in which the at least one OCSA sequesters one or more odorous compounds via absorption, adsorption, coordinate bonds, covalent bonds, or any combination thereof. Absorbent articles including such films (e.g., as a backsheet) are also provided.

### BACKGROUND

Absorbent articles, such as pads, find widespread use in absorbing body or bodily fluids in articles such as catamenial devices, diapers (both for babies and individuals with incontinence problems), sanitary napkins, tampons, wound dressings, and bandages. Such absorbent articles may incorporate super absorbent polymers that absorb many times their own weight of fluid or other fibrous materials, such as cotton, wood pulp, and paper. The bodily fluids absorbed by such absorbent members may include vomit, blood, pus, sweat, semen, secretions, menstrual discharge, urine, and fecal matter. In this regard, the bodily fluid may have an unpleasant odor (malodor) due to odor-causing molecules which may be aliphatic, aromatic, or heterocyclic compounds containing oxygen, sulfur, or nitrogen. The odor-causing molecules can be masked using a more pleasant smelling molecule, such as a perfume.

There remains a need in the art, however, for films, which may be incorporated into a variety of absorbent article, capable of sequestering (e.g., neutralize and/or destroy) one or more odor-causing molecules that are generated/released over an extended period of time.

### SUMMARY OF INVENTION

One or more embodiments of the invention may address one or more of the aforementioned problems. Certain embodiments according to the invention provide a film, such as a breathable film comprising a vapor-permeable and liquid impermeable (VPLI) film. The VPLI may comprise a microporous film. The microporous film may include a plurality of micropores that define or otherwise provide a tortuous path for the flow of vapors to pass therethrough while simultaneously providing desirable liquid barrier properties. The microporous film may include (a) a first outermost surface, (b) a second outermost surface, (c) a thickness extending between the first outermost surface and the second outermost surface, and (d) at least one odorous compound sequestering agent (OCSA) dispersed throughout the thickness of the film, wherein the at least one OCSA comprises (i) at least one salt of ricinoleic acid (e.g., zinc ricinoleate), and/or (ii) one or more cucurbituril compounds, such as CB[5], CB[6], CB[7], CB[8], or any mixture thereof, and/or (iii) one or more zeolites, and/or (iv) one or more halo active aromatic sulfonamide compounds.

In another aspect, the present invention provides a method of forming a breathable film, such as those described and disclosed herein. The method may comprise the following: (i) forming a polymer melt; (ii) adding a pore-forming filler material to the polymer melt; (iii) adding a dry masterbatch to the polymer melt, wherein the dry masterbatch comprises at least one odorous compound sequestering agent (OCSA) comprising (a) at least one salt of ricinoleic acid (e.g., zinc ricinoleate), and/or (b) one or more cucurbituril compounds, such as CB[5], CB[6], CB[7], CB[8], or any mixture thereof, and/or (c) one or more zeolites, and/or (d) one or more halo active aromatic sulfonamide compounds; (iv) admixing the pore-forming filler material and the dry masterbatch into the polymer melt; (v) melt extruding the polymer melt including the pore-forming filler material and at least one OCSA to form an intermediate film; and (vi) incrementally stretching the intermediate film in a machine-direction and/or a cross-direction to form the breathable film.

In another aspect, the present invention provides an absorbent article comprising: (i) a liquid permeable topsheet (LPTS), such as a nonwoven fabric and/or a top-apertured-film (TAF); (ii) a backsheet comprising a film, such as a breathable film as described and disclosed herein; and (iii) an absorbent core, wherein the absorbent core is located directly or indirectly between the LPTS and the backsheet.

In yet another aspect, the present invention provides a method of making an absorbent article comprising the following: (i) providing or forming a liquid permeable topsheet (LPTS), such as a nonwoven fabric and/or a top-apertured-film (TAF); (ii) providing or forming a backsheet comprising a film, such as a breathable film as described and disclosed herein; (iii) providing or forming an absorbent core, wherein the absorbent core is located directly or indirectly between the LPTS and the backsheet; and (iv) bonding the backsheet directly or indirectly to the absorbent core.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates an absorbent article in which a backsheet is directly bonded to an absorbent core in accordance with certain embodiments of the invention;

FIG. 2 illustrates an absorbent article in which a backsheet is indirectly bonded to an absorbent core via an adhesive layer in accordance with certain embodiments of the invention;

FIG. 3 illustrates an absorbent article including an acquisition distribution layer located between a topsheet and an absorbent core in accordance with certain embodiments of the invention;

FIGS. 4-10 illustrate visual comparison of films investigating the impact of odor control master batch, calcium carbonate (CaCO₃) filler, and activation on urine odor reduction; and

FIGS. 11-13 illustrate visual comparison of films investigating the necessity of an odor control master batch in conjunction with calcium carbonate (CaCo₃) filler, and activation for effective odor reduction.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to films, such as breathable films or non-breathable films, incorporating odor control functionality. Breathable films, in accordance with certain embodiments of the invention, may comprise a microporous film having a plurality of micropores that define or otherwise provide a tortuous path for vapor to pass through the breathable film while simultaneously providing a desirable level of liquid barrier properties. The odor control functionality, for example, may be provided by the incorporation of one or more odorous compound sequestering agents (OCSA) dispersed throughout the thickness of the breathable film. The one or more OCSAs, for instance, may be incorporated into the breathable film as a melt additive incorporated into the film-forming melt (e.g., polymer melt) used to produce the breathable film. For example, the one or more OCSAs may be a component of a dry (e.g., pellets) masterbatch comprising a polymeric carrier (e.g. a polymer matrix) and the one or more OCSAs dispersed throughout the polymeric carrier. The dry masterbatch, for instance, may be easily added to the polymer melt used for forming the film. In this regard, the one or more OCSAs may be directly incorporated into the polymeric film formulation of the breathable film. As noted above, the breathable film may comprise a microporous film that provides increased surface area (e.g., via the plurality of micron-sized pores and/or micron-sized channels) for minimizing malodor in a soiled absorbent article. For example, the passing of odorous gaseous compounds through the microporous film layer ensures a tortuous path having a high surface area for the odorous gaseous compounds to interact with the one or more OCSAs incorporated into the microporous film. Stated somewhat differently, such embodiments may provide a greater interaction between odorous gaseous compounds and the one or more OCSAs present throughout the body or thickness of the microporous film (or layer of the film).

Accordingly, a more efficient use of a given amount of OCSA(s) may be realized as compared to incorporating one or more OCSAs into, for example, an absorbent core of an absorbent article. In accordance with certain embodiments of the invention, however, one or more OCSAs may also be added to the absorbent core and/or an acquisition distribution layer (ADL) if desired.

The terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The term “layer”, as used herein, may comprise a generally recognizable combination of similar material types and/or functions existing in the X-Y plane.

The term “machine direction” or “MD”, as used herein, comprises the direction in which the film and/or fabric produced or conveyed. The term “cross-direction” or “CD”, as used herein, comprises the direction of the film and/or fabric substantially perpendicular to the MD.

The term “film,” as used herein, may comprise a polymeric or elastomeric layer or layers made using a film extrusion process, such as a cast film or blown film extrusion process. This term may also include films rendered microporous by mixing polymer and/or elastomer with filler, forming a film from the mixture, and optionally stretching the film.

The term “microporous” film, as used herein, may comprise films or membranes having a narrow pore sized distribution in the submicron range, from 1.0 to 10 microns. The microporous films can be made by a number of processes, which include (a) dissolving polymers in solution followed by extraction of the solvent by water vapor, (b) stretching of crystallizable polymers which results in microsized tears, and (c) stretching of a mineral filled polyolefin film. The polymers used in the microporous films include PTFE, polyolefins, polyurethanes, polyamides, and polyesters.

The term “filler”, as used herein, may comprise particles or aggregates of particles and other forms of materials that can be added to a polymeric film blend. According to certain embodiments of the invention, a filler may not substantially chemically interfere with or adversely affect the extruded film. According to certain embodiments of the invention, the filler is capable of being uniformly dispersed throughout the film or a layer comprised in a multilayer film. Fillers, for example, may comprise particulate inorganic materials such as, for example, calcium carbonate (CaCo₃), various kinds of clay, silica, alumina, barium sulfate, sodium carbonate, talc, magnesium sulfate, titanium dioxide, zeolites, aluminum sulfate, cellulose-type powders, diatomaceous earth, magnesium sulfate, magnesium carbonate, barium carbonate, kaolin, mica, carbon, calcium oxide, magnesium oxide, aluminum hydroxide, glass particles, and the like, and organic particulate materials such as high-melting point polymers (e.g., TEFLON® and KEVLAR® from E.I. DuPont de Nemours and Company), pulp powder, wood powder, cellulose derivatives, chitin and chitin derivatives, and the like. Filler particles may optionally be coated with a fatty acid, such as stearic acid or reduced stearic acid, or a larger chain fatty acid, such as behenic acid. Without intending to be bound by theory, coated filler particles may facilitate the free flow of the particles (in bulk) and their case of dispersion into the polymer matrix, according to certain embodiments of the invention.

As used herein, the term “monolithic” film may comprise any film that is continuous and substantially free or free of pores (e.g., devoid of pores). In certain alternative embodiments of the invention, a “monolithic” film may comprise fewer pore structures than would otherwise be found in a microporous film. According to certain non-limiting exemplary embodiments of the invention, a monolithic film may act as a barrier to liquids and particulate matter but allow water vapor to pass through, such as by absorbing water vapor on one side of the film, transporting the water vapor through the film, and releasing the water vapor on the opposite side of the film.

The term “highly breathable polymer”, as used herein, may comprise any polymer or elastomer that is selectively permeable to water vapor but substantially impermeable to liquid water and that can form a breathable film, for example, in which the polymer is capable of absorbing and desorbing water vapor and providing a barrier to aqueous fluids (e.g., water, blood, etc.). For example, a highly breathable polymer can absorb water vapor from one side of a film and release it to the other side of film, thereby allowing the water vapor to be transported through the film. As the highly breathable polymer can impart breathability to films, films formed from such polymers do not need to include pores (e.g., monolithic film). According to certain embodiments of the invention, “highly breathable polymer” may comprise any thermoplastic polymer or elastomer having a moisture vapor transmission rate (MVTR) of at least 500 g/m²/day when formed into a film. According to certain embodiments of the invention, “highly breathable polymer” may comprise any thermoplastic polymer or elastomer having a MVTR of at least 750 g/m²/day or of at least 1000 g/m²/day when formed into a film, such as a film having, for example, a thickness of about 25 microns or less. According to certain embodiments of the invention, highly breathable polymers may comprise, for example, any one or combination of a polyether block amide copolymer (e.g., PEBAX® from Arkema Group), polyester block amide copolymer, copolyester thermoplastic elastomer (e.g., ARNITEL® from DSM Engineering Plastics, HYTREL® from E.I. DuPont de Nemours and Company), or thermoplastic urethane elastomer (TPU). Highly breathable polymers may be utilized to form a monolithic film.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, the aldehyde group-CHO is attached through the carbon of the carbonyl group.

The term “alkyl”, as used herein, refers to a radical composed entirely of carbon atoms and hydrogen atoms which is fully saturated. The alkyl radical may be linear, branched, or cyclic, and such radicals may be referred to as linear alkyl, branched alkyl, or cycloalkyl.

The term “aromatic”, as used herein, refers to a radical that has a ring system containing a delocalized conjugated pi system with a number of pi-electrons that obeys Hückel's Rule. The ring system may include heteroatoms (e.g. N, S, Se, Si, O), or may be composed exclusively of carbon and hydrogen. Exemplary aromatic groups include phenyl, thienyl, naphthyl, and biphenyl.

The term “aryl”, as used herein, refers to an aromatic radical composed exclusively of carbon and hydrogen. Exemplary aryl groups include phenyl, naphthyl, and biphenyl.

The term “heteroaryl”, as used herein, refers to an aromatic radical containing at least one heteroatom. Exemplary heteroaryl groups include thienyl. Note that “heteroaryl” is a subset of “aromatic”, and is exclusive of “aryl”.

The term “alkoxy”, as used herein, refers to an alkyl radical which is attached to an oxygen atom, e.g., —O—CₙH₂ₙ₊₁, to a molecule containing such a radical.

The term “halogen”, as used herein, refers to fluorine, chlorine, bromine, and iodine.

The term “substituted”, as used herein, refers to at least one hydrogen atom on the named radical being substituted with another functional group, such as halogen, —CN, or —NO2. Besides the aforementioned functional groups, an aromatic group may also be substituted with alkyl or alkoxy. An exemplary substituted aryl group is methylphenyl.

The term “alkali metal”, as used herein, refers to lithium, sodium, and potassium.

The term “alkaline earth metal”, as used herein, refers to magnesium and calcium.

In one aspect, certain embodiments according to the invention provide a breathable film comprising a vapor-permeable and liquid impermeable (VPLI) film comprising a microporous film. The microporous film may include a plurality of micropores that define or otherwise provide a tortuous path for the flow of vapors to pass therethrough while simultaneously providing desirable liquid barrier properties. Microporous films may generally be produced by dispersing finely divided particles of a non-hygroscopic filler material, such as an inorganic salt (e.g., calcium carbonate), into a suitable polymer followed by forming a film of the filled polymer and stretching the film to provide good porosity and water vapor absorption or transmission. In accordance with certain embodiments of the invention, the microporous film may comprise a polyolefin, such as a polyethylene or a polypropylene, or a copolymer comprising a first polyolefin, such as a first polyethylene, and a second polyolefin, such as a second polypropylene. The microporous film may include (a) a first outermost surface, (b) a second outermost surface, (c) a thickness extending between the first outermost surface and the second outermost surface, and (d) at least one odorous compound sequestering agent (OCSA) dispersed throughout the thickness of the film, wherein the at least one OCSA comprises (i) at least one salt of ricinoleic acid (e.g., zinc ricinoleate), and/or (ii) one or more cucurbituril compounds, such as CB[5], CB[6], CB[7], CB[8], or any mixture thereof, and/or (iii) one or more zeolites, and/or (iv) one or more halo active aromatic sulfonamide compounds.

In accordance with certain embodiments of the invention, the one or more halo active aromatic sulfonamide compounds (if present) may optionally have a structure according to Formula (I):

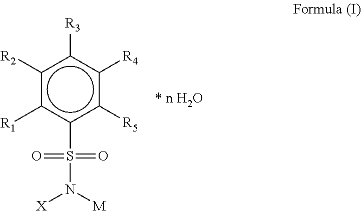

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen;
M is an alkali or alkaline earth metal; and
n is the number of water molecules per molecule of the sulfonamide compound

Generally, M is sodium or potassium. X is generally chlorine, bromine, fluorine, or iodine, and in particular embodiments is chlorine. Compounds of Formula (I) may or may not be hydrated, as indicated by the variable n. In particular embodiments, the compounds of Formula (1) are a trihydrate (i.e., n=3) or a hexahydrate (i.e. n=6). In other embodiments, the compound is in a solid form, such as a powder.

When the phenyl and/or alkyl group is substituted, one or more hydrogen atoms may be independently replaced with hydroxyl or halogen.

In particular embodiments of Formula (I), R₃ is methyl, COOH, or COOM₁; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; M₁ is an alkali or alkaline earth metal; and n is the number of water molecules per molecule of the sulfonamide compound.

In further embodiments, R₃ is methyl, COOH, or COOM₁; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; M is an alkali or alkaline earth metal; n is the number of water molecules per molecule of the sulfonamide compound; and at least one of R₁, R₂, R₄, and R₅ is not hydrogen.

In yet other embodiments of Formula (I), R₃ is selected from COOH, COOM₁, COOR′, CON(R″)₂, CN, NO₂, halogen, and substituted or unsubstituted C₂-C₁₂ alkyl; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; M is an alkali or alkaline earth metal; and n is the number of water molecules per molecule of the sulfonamide compound.

In still other embodiments of Formula (I), R₁, R₂, R₃, R₄, and R₅ are independently selected from hydrogen, COOH, COOM₁, NO₂, halogen, N(R″)₂, substituted or unsubstituted C₁-C₁₂ alkyl, and substituted or unsubstituted aromatic; X is halogen; M is an alkali or alkaline earth metal; and n is the number of water molecules per molecule of the sulfonamide compound.

In yet other embodiments of Formula (D), R₂ and R₄ are identical to each other; and R₁, R₃, and R₅ are hydrogen.

In yet other embodiments of Formula (I), R₂ and R₄ are hydrogen; and R₁, R₃, and R₅ are identical to each other.

In more specific embodiments of Formula (I), R₃ is selected from COOH, COOM₁, COOR′, and CON(R″)₂. Most desirably, R₃ is COOH or COOM₁, while R₁, R₂, R₄, and R₅ are hydrogen.

In other embodiments of Formula (I), R₁, R₂, R₃, R₄, and R₅ are independently selected from hydrogen, COOH, COOM₁, COOR′, CON(R″)₂, NO₂, halogen, N(R″)₂, substituted or unsubstituted C₁-C₁₂ alkyl, and substituted or unsubstituted aromatic; wherein at least one of R₁, R₂, R₃, R₄, and R₅ is not hydrogen; X is halogen; M is an alkali or alkaline earth metal; and n is the number of water molecules per molecule of the sulfonamide compound.

In still other embodiments of Formula (I), R₃ is COOH or COOM₁; R₁, R₂, R₄, and R₅ are independently selected from hydrogen. NO₂, halogen, N(R″)₂, substituted or unsubstituted C₁-C₁₂ alkyl, and substituted or unsubstituted aromatic; X is halogen; M is an alkali or alkaline earth metal; and n is the number of water molecules per molecule of the sulfonamide compound. In further specific embodiments, at least one of R₁, R₂, R₄, and R₅ is not hydrogen.

In some embodiments of Formula (I), at least one of R₁, R₂, R₃, R₄, or R₅ are not hydrogen. In more specific embodiments of Formula (I), at least two of R₁, R₂, R₃, R₄, or R₅ are not hydrogen. In other words, the benzene ring contains the sulfonamide substituent and an additional one or two other substituents.

In other embodiments of Formula (I), the halo active aromatic sulfonamide compound has the structure of Formula (II):

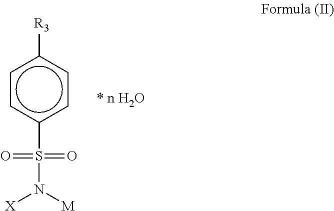

wherein R₃ is COOR′, R′ is hydrogen, an alkali metal, an alkaline earth metal, substituted C₁-C₁₂ alkyl, unsubstituted C₁-C₇ alkyl, substituted aromatic, or unsubstituted aromatic;
X is halogen: M is an alkali or alkaline earth metal; and n is the number of water molecules per molecule of the sulfonamide compound. The N-chloro-4-carboxybenzenesulfonamide compound of Formula (II) is also referred to herein as BENZ. BENZ exhibits a lower chlorine smell than chloramine-T or chloramine-B. When BENZ is combined with at least one fragrance, there is no detectable chlorine smell for most humans.

Two particular sulfonamide compounds contemplated for use are N-chloro-p-toluenesulfonamide (i.e. chloramine-T) and N-chloro-4-carboxybenzenesulfonamide (i.e. BENZ). These two compounds are shown below as Formulas (111) and (IV):

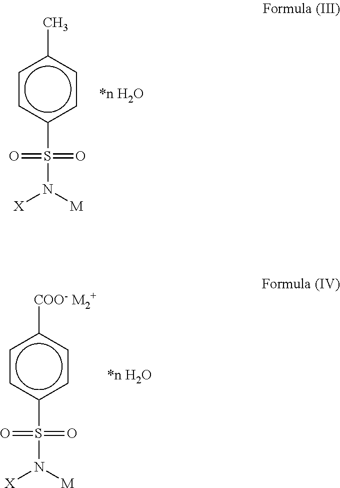

wherein M₂ is hydrogen, an alkali metal, or an alkali earth metal; X is halogen, M is independently an alkali or alkaline earth metal; and n is the number of water molecules per molecule of each sulfonamide compound. Desirably, M₂ is hydrogen, sodium, or potassium.

In other particular embodiments, one or more of R₁, R₂, R₃, R₄, and R₅ are substituted with —COOR′ (and the others are hydrogen). In this regard, it is believed that when the halo active aromatic sulfonamide compound has two or more ionic charges, that the compound has higher antimicrobial performance. The antimicrobial performance of these compounds of Formula (I) was not expected, because sulfonamide groups having a halogen atom bonded to the nitrogen atom are not present in molecules having known antimicrobial properties.

The halo active aromatic sulfonamide compounds of base Formula (I) are stable and do not decompose in aqueous solution, allowing the disinfecting composition to have a long shelf life. The compounds of Formula (I) are also very soluble in water, low in toxicity, and have minimal bleach odor.

In accordance with certain embodiments of the invention, the one or more halo active aromatic sulfonamide compounds may comprise from about 0.0001 wt. % to about 40 wt. % of the microporous film, such as at least about any of the following: 0.0001, 0.001, 0.01, 0.02, 0.05, 0.08, 0.1, 0.5, 0.2, 0.5, 0.8, 1, 2, 5, 8, and 10 wt. % of the microporous film, and/or at most about any of the following: 40, 30, 20, 18, 15, 12, and 10 wt. % of the microporous film.

The one or more cucurbituril compounds, if present at all, refer to macrocyclic molecules made of glycoluril (═C₄H₂N₄O₂═) monomers linked by methylene bridges (—CH₂—). Cucurbiturils, in this regard, are members of the cavitand family, and the general cucurbituril structure is based on the cyclic arrangement of glycoluril subunits linked by methylene bridges, in which the oxygen atoms are located along the edges of the band and are tilted inwards, forming a partly enclosed cavity (cavitand). The name is derived from the resemblance of these molecules with a pumpkin of the family of Cucurbitaceae. Cucurbiturils are commonly written as cucurbit[n]uril, where n is the number of glycoluril units. Two common abbreviations are CB[n], or simply CBn. For example, cucurbit[8]uril (CB[8]; CAS 259886-51-6) is a barrel shaped container molecule which has eight repeat glycoluril units and an internal cavity volume of 479A3 (see structure below). CB[8] is readily synthesized using standard techniques and is available commercially (e.g. Sigma-Aldrich, MO USA). CB[8] is illustrated as follows:

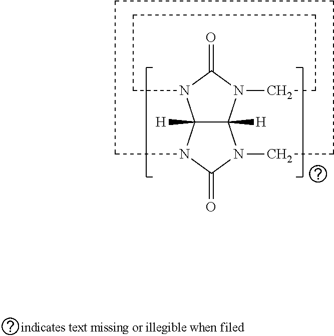

In accordance with certain embodiments of the invention, one or more cucurbituril compounds may be incorporated into the body or thickness of the microporous film where they may be fee to form a complex with a malodour molecule. The one or more cucurbituril compounds may comprise a CB[5], CB[6], CB[7], CB[8], CB[9], CB[10], CB[11], CB[12], CB[13], CB[14] compound, or mixtures thereof. For instance, the one or more cucurbituril compounds may comprise a mixture of different sized (e.g., cavity) cucurbituril compounds. By way of example only, the one or more cucurbituril compounds may comprise a mixture of at least two different cucurbiturils selected from CB[5], CB[6], CB[7] and CB[8].

In accordance with certain embodiments of the invention, the one or more cucurbituril compounds, if present, may comprise from about 0.1 to about 99% by weight of CB [5] based on a total cucurbituril weight, such as at least about any of the following: 0, 1, 1, 3, 5, 8, 10, 20, 30, 40, and 50% by weight, and/or at most about any of the following: 99, 95, 90, 80, 70, 60, and 50% by weight. Additionally or alternatively, the one or more cucurbituril compounds, if present, may comprise from about 0.1 to about 99% by weight of CB[6] based on a total cucurbituril weight, such as at least about any of the following: 0.1, 1, 3, 5, 8, 10, 20, 30, 40, and 50% by weight, and/or at most about any of the following: 99, 95, 90, 80, 70, 60, and 50% by weight. Additionally or alternatively, the one or more cucurbituril compounds, if present, may comprise from about 0.1 to about 99% by weight of CB[7] based on a total cucurbituril weight, such as at least about any of the following: 0.1, 1, 3, 5, 8, 10, 20, 30, 40, and 50% by weight, and/or at most about any of the following: 99, 95, 90, 80, 70, 60, and 50% by weight. Additionally or alternatively, the one or more cucurbituril compounds, if present, may comprise from about 0.1 to about 99% by weight of CB[8] based on a total cucurbituril weight, such as at least about any of the following: 0.1, 1, 3, 5, 8, 10, 20, 30, 40, and 50% by weight, and/or at most about any of the following: 99, 95, 90, 80, 70, 60, and 50% by weight. Additionally or alternatively, the one or more cucurbituril compounds, if present, may comprise a total concentration or total cucurbituril weight and the CB[5] and/or CB[6] and/or CB[7] and/or CB[8] may collectively be greater than 75% by weight, more particularly greater than about 90% by weight, more particularly greater than about 99% by weight of the total cucurbituril weight. The remaining components of the cucurbituril mixture may contain CB[4], CB[9] and/or higher cucurbiturils (i.e. CB[101-CB[201), either as a single sized cucurbituril or as a mixture of these sizes.

The one or more cucurbituril compounds, if present at all, may have an average particle size from about 0.01 microns to about 50 microns, such as at least about any of the following: 0.01, 0.5, 1, 3, 5, 8, and 10 microns, and/or at most about any of the following: 50, 40, 30, 20, and 10 microns. Additionally or alternatively, the one or more cucurbituril compounds are present in an uncomplexed form.

In accordance with certain embodiments of the invention, one or more salts of ricinoleic acid, such as zinc ricinoleate, may be incorporated into the body or thickness of the microporous film where they may be free sequester a malodor molecule. The one or more salts of ricinoleic acid may comprise a transition metal salt (e.g., copper) of ricinoleic acid and/or a post-transition metal salt (e.g., zinc, aluminum, etc.) of ricinoleic acid. By way of example only, the salts of ricinoleic acid may comprise a mixture of at least two different salts of ricinoleic acid. Alternatively, all of the salts of ricinoleic acid may be one specific salt, such as zinc ricinoleate. Additionally or alternatively, the OCSA may comprise a salt of other fatty acids, such as those found in vegetable oils (e.g., castor oil, which includes oleic acid, linoleic acid, stearic acid and palmitic acid).

In accordance with certain embodiments of the invention, the one or more salts of ricinoleic acid, may comprise from about 0.1 to about 100% by weight of zinc ricinoleate based on a total weight associated with salts of rincinoleic, such as at least about any of the following: 0.1, 1, 3, 5, 8, 10, 20, 30, 40, and 50% by weight, and/or at most about any of the following: 100, 99, 95, 90, 80, 70, 60, and 50% by weight. In accordance with certain embodiments of the invention, the one or more salts of ricinoleic acid (e.g., zinc ricinoleate) may comprise from about 0.0001 wt. % to about 40 wt. % of the microporous film, such as at least about any of the following: 0.0001, 0.001, 0.01, 0.02, 0.05, 0.08, 0.1, 0.5, 0.2, 0.5, 0.8, 1, 2, 5, 8, and 10 wt. % of the microporous film, and/or at most about any of the following: 40, 30, 20, 18, 15, 12, and 10 wt. % of the microporous film.

In accordance with certain embodiments of the invention, one or more zeolites may be incorporated into the body or thickness of the film, such as a microporous film, where they may be free sequester a malodor molecule. The one or more zeolites may comprise a microporous material (e.g., crystalline aliminosilicate) including, for example, silicon, aluminum, and oxygen atom(s). The one or more zeolites may include a counter ion, such as a metal ion or H⁺. The one or more zeolites may have a variety of framework structures, for example, such as 3A, 4A, 5A LTA, MOR, HEU, and ANA-types. By way of example only, the one or more zeolite may comprise diatomaceous earth. The one or more zeolites may have an average particle size, based on the largest cross-sectional dimension, from about 1 to about 200 microns, such as at least about any of the following: 1, 3, 5, 8, 10, 20, 40, and 50 microns, and/or at most about any of the following: 200, 150, 100, 75, and 50 microns. Additionally or alternatively, the one or more zeolites may include a plurality of pores defined throughout the individual zeolite particles. In this regard, the one or more individual zeolite particles may have an average pore size from 1 nm to 100 nm, such as at least about any of the following: 1, 2, 3, 5, 8, 10, 15, 20, 25, 30, 40, and 50 nm, and/or at most about any of the following: 100, 90, 80, 70, 60, and 50 nm. Additionally or alternatively, in accordance with certain embodiments of the invention the one or more zeolites may comprise from about 0.0001 wt. % to about 40 wt. % of the film, such as a microporous film, such as at least about any of the following: 0.0001, 0.001, 0.01, 0.02, 0.05, 0.08, 0.1, 0.5, 0.2, 0.5, 0.8, 1, 2, 5, 8, and 10 wt. % of the film, such as microporous film, and/or at most about any of the following: 40, 30, 20, 18, 15, 12, and 10 wt. % of the film, such as microporous film.

In accordance with certain embodiments of the invention, the at least one OCSA comprises a mixture of (i) one or more salts of ricinoleic acid, such as zinc ricinoleate, (ii) one or more cucurbituril compounds, and/or (iii) one or more zeolites, and/or (iv) one or more halo active aromatic sulfonamide compounds, such as according to Formula (I), wherein the salts of ricinoleic acid comprises from about 1 to 99% by weight of a total weight of the at least one OCSA, such as at least about any of the following: 0.1, 1, 3, 5, 8, 10, 20, 30, 40, and 50% by weight, and/or at most about any of the following: 99, 95, 90, 80, 70, 60, and 50% by weight, and/or wherein the one or more cucurbituril compounds comprises from about 1 to 99% by weight of a total weight of the at least one OCSA, such as at least about any of the following: 0.1, 1, 3, 5, 8, 10, 20, 30, 40, and 50% by weight, and/or at most about any of the following: 99, 95, 90, 80, 70, 60, and 50% by weight, and/or wherein the one or more zeolites comprises from about 1 to 99% by weight of a total weight of the at least one OCSA, such as at least about any of the following: 0.1, 1, 3, 5, 8, 10, 20, 30, 40, and 50% by weight, and/or at most about any of the following: 99, 95, 90, 80, 70, 60, and 50% by weight, and/or wherein the one or more halo active aromatic sulfonamide compounds, such as of Formula (I), comprises from about 1 to 99% by weight of a total weight of the at least one OCSA, such as at least about any of the following: 0.1, 1, 3, 5, 8, 10, 20, 30, 40, and 50% by weight, and/or at most about any of the following: 99, 95, 90, 80, 70, 60, and 50% by weight. The total weight of the at least one OCSA does not exceed 100%.

In accordance with certain embodiments of the invention, the at least one OCSA may also comprise a plurality of nanoparticles having at least one metal ion adsorbed thereon, such as those described in U.S. Pat. No. 7,794,737, the contents of which are hereby incorporated by reference. The plurality of nanoparticles, for example, may have an average diameter from about or less, such as at most about any of the following: 500, 400, 300, 200, and 100 nm, and/or at least about any of the following: 1, 10, 20, 40, 60, 80, and 100 nm. In accordance with certain embodiments of the invention, the at least one metal ion may be selected from the group consisting of a copper ion, a silver ion, a gold ion, an iron ion, and combinations thereof. Additionally or alternatively, the plurality of nanoparticles comprise a substrate to which the at least one metal ion is adsorbed, the substrate comprising silica, alumina, magnesium oxide, titanium dioxide, iron oxide, gold, zinc oxide, copper oxide, organic nanoparticles such as polystyrene, and combinations thereof. Additionally or alternatively, the plurality of nanoparticles comprises an average surface area from at least about 100 m²/g, such as at least about any of the following: 100, 200, 300, and 500 m²/g, and/or at most about any of the following: 2000, 1500, 1000, 800, 600, and 500 m²/g.

In accordance with certain embodiments of the invention, the microporous film comprises a pore-forming filler material comprising a plurality of particles about which the plurality of micropores are formed. As described above, the pore-forming filler (e.g., “filler” above) may comprise a wide variety of materials. In accordance with certain embodiments of the invention, the pore forming material (e.g., filler) may comprise from about 3 to about 60% by weight of the microporous film, such as at least about any of the following: 3, 5, 8, 10, 12, 15, 18, 20, 22, 25, 28, and 30% by weight of the microporous film, and/or at most about any of the following: 60, 55, 50, 45, 40, 35, and 30% by weight of the microporous film. As noted above, the pore-forming filler (e.g., filler) may comprise, for example, calcium carbonate, which has a chemical formula of CaCO₃.

As noted above, the at least one OCSA (regardless of type or combination of OCSAs) sequesters one or more odorous compounds, such as one or more carboxylic acids, such a C₁-Cs carboxylic acids (e.g., propanoic acid, butanoic acid, and pentanoic acid); sulfur-containing compounds, such as methanethiol and hydrogen sulfide; and nitrogen-containing compounds, such as indole, skatole, ammonia, trimethylamine, and urea. In accordance with certain embodiments of the invention, the at least one OCSA sequesters the one or more odorous compounds via absorption, adsorption, coordinate bonds, covalent bonds, or any combination thereof.

In accordance with certain embodiments of the invention, the film may have a basis weight from about 5 to about 100 gsm, such as at least about any of the following: 5, 8, 10, 12, 15, 18, 20, 22, 25, 28, and 30 gsm, and/or at most about any of the following: 100, 90, 80, 70, 60, 50, 40, and 30 gsm.

In accordance with certain embodiments of the invention, the microporous film may have a moisture vapor transmission rate (MVTR) from about 200 to about 20,000 g/m²/24 hours as determined according to EDNA/INDA Worldwide Strategic Method: WSP 70.4(08), such as at least about any of the following: 200, 220, 250, 280, 300, 320, 350, 380, 400, 420, 450, 500, 600, 700, 800, 900, 1000, 1200, 1400, 1500, 1600, 1800, 2000, 2500, 3000, 3500, 4000, 4500, 5000, 6000, 7000, 8000, 9000, and 10,000 g/m²/24 as determined according to WSP 70.4(08), and/or at most about any of the following: 20,000, 19,000, 18,000, 17,000, 16,000, 15,000, 14,000, 13,000, 12,000, 11,000, and 10,000 g/m²/24 as determined according to WSP 70.4(08).

In accordance with certain embodiments of the invention, the microporous film comprises a melt-extruded film. Additionally or alternatively, the microporous film has been subjected to incremental stretching in a machine direction and/or a cross-direction. The incremental stretching, for instance, may facilitate the formation of the plurality of micropores to enhance vapor breathability.

In accordance with certain embodiments of the invention, the microporous film may further comprise a coating adjacent the first outermost surface of the microporous film, the second outermost surface of the microporous film, or both. The coating may comprise at least one OCSA comprising one or more salts of a fatty acid(s), such as ricinoleic acid, dispersed throughout the coating, and/or one or more halo active aromatic sulfonamide compounds of Formula (I) dispersed throughout the coating and/or one or more cucurbituril compounds dispersed throughout the coating. For example, the coating may comprise from about 0.0001 wt. % to about 50 wt. % of the one or more salts of a fatty acid(s), such as ricinoleic acid, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more salts of a fatty acid(s), such as ricinoleic acid, and/or at most about any of the following: 50, 40, 30 and 25 wt. % of the one or more salts of a fatty acid(s), such as ricinoleic acid. Additionally or alternatively, the coating may comprise from about 0.0001 wt. % to about 50 wt. % of the one or more halo active aromatic sulfonamide compounds of Formula (I), such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more halo active aromatic sulfonamide compounds of Formula (I), and/or at most about any of the following: 50, 40, 30 and 25 wt. % of the one or more halo active aromatic sulfonamide compounds of Formula (I). Additionally or alternatively, the coating may comprise from about 0.0001 wt. % to about 50 wt. % of the one or more cucurbituril compounds, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more cucurbituril compounds, and/or at most about any of the following: 50, 40, 30 and 25 wt. % of the one or more cucurbituril compounds. Additionally or alternatively, the coating may have a coating-thickness from about 10 microns to about 2000 microns, such as at least about any of the following: 10, 15, 20, 25, 30, 35, 40, 45, 50, 80, 100, 150, 200, 250, 300, 500, 800, and 1000 microns, and/or at most about any of the following: 2000, 1800, 1500, 1200, and 1000 microns.

The coating, if present, may be formed onto the microporous film from a coating composition that that may optionally include the one or more OCSAs or be devoid of one or more OCSAs, an evaporative solvent (e.g., water, an alcohol, or an organic solvent), and a binder component (e.g., polymeric resins, cellulose or cellulose derivatives). The coating may be formed by allowing the coating composition after being applied to the microporous film to evaporate over a period of time. That is, after application of the coating composition to the microporous film, the solvent may be allowed to evaporate, thereby forming a coating (e.g., a dry coating) on the microporous film. The coating composition can be applied to one or more surfaces of the microporous film by simple solution, spin coating, dip coating, contour coating, doctor blading, solution casting, extrusion/dispersion coating, spray coating, gravure coating, printing techniques such as screen printing, ink-jet printing and the like, electrostatic application, fogging, wipes, misting, or immersion, amongst other applications.

In accordance with certain embodiments of the invention, the film may not comprise a breathable film (e.g., microporous and/or monolithic film). Instead, the film may comprise a non-breathable film. In this regard, the non-breathable film may include the same properties and/or additives (e.g., OCSA) in the values noted above when discussing microporous films (e.g., breathable films) with the exception of the moisture vapor transmission rate (MVTR). In accordance with certain embodiments of the invention, for example, the MVTR of the non-breathable film may have a MVTR below about 200 g/m²/24 hours as determined according to EDNA/INDA Worldwide Strategic Method: WSP 70.4(08), such as at least about any of the following: 0, 5, 10, 20, 30, 40, and 50 g/m²/24 as determined according to WSP 70.4(08), and/or at most about any of the following: 200, 150, 100, 75, and 50 g/m²/24 as determined according to WSP 70.4(08).

In another aspect, the present invention provides a method of forming a breathable film, such as those described and disclosed herein. The method may comprise the following: (i) forming a polymer melt; (ii) adding a pore-forming filler material to the polymer melt; (iii) adding a dry masterbatch to the polymer melt, wherein the dry masterbatch comprises at least one odorous compound sequestering agent (OCSA) comprising (a) at least one salt of ricinoleic acid (e.g., zinc ricinoleate), (b) one or more cucurbituril compounds, such as CB[5], CB[6], CB[7], CB[8], or any mixture thereof, and/or (c) one or more zeolites, and/or (d) one or more halo active aromatic sulfonamide compounds; (iv) admixing the pore-forming filler material and the dry masterbatch into the polymer melt; (v) melt extruding the polymer melt including the pore-forming filler material and at least OCSA to form an intermediate film; and (vi) incrementally stretching the intermediate film in a machine-direction and/or a cross-direction to form the breathable film. In accordance with certain embodiments, the method may comprise forming a non-breathable film, in which the addition of the pore-forming filler may be excluded from the foregoing steps. Additionally or alternatively, a method of forming a non-breathable film may exclude the step of incrementally stretching the film. In this regard, the method may provide a non-breathable film that is devoid or substantially devoid of micropores.

In accordance with certain embodiments of the invention, the dry masterbatch includes a polymer matrix component (e.g., a carrier) and the at least one OCSA is dispersed throughout the polymer matrix. For example, the polymer matrix may comprise a matrix polymer corresponding to a polymer component of the polymer melt. By way of example only, the polymer melt may include a first polypropylene or first polyethylene, and the matrix polymer may comprise a corresponding second polypropylene or second polyethylene.

In another aspect, the present invention provides an absorbent article comprising: (i) a liquid permeable topsheet (LPTS), such as a nonwoven fabric and/or a top-apertured-film (TAF); (ii) a backsheet comprising a film, such as a breathable film or non-breathable film as described and disclosed herein; and (iii) an absorbent core, wherein the absorbent core is located directly or indirectly between the LPTS and the backsheet. The LPTS, in accordance with certain embodiments of the invention may comprise a TAF formed from a film, such as any of those including one or more OCSA(s) as described and disclosed herein. In accordance with certain embodiments of the invention, the absorbent article may further comprise an acquisition distribution layer (ADL) located directly or indirectly between the LPTS and the absorbent core. The ADL, for example, may comprises an apertured-film, a high-loft nonwoven fabric, or a combination thereof. Additionally or alternatively, the ADL comprises an apertured-film that includes a second group of one or more OCSAs dispersed throughout a thickness of the apertured-film, applied to a top surface of the apertured film, and/or applied to a bottom surface of the apertured-film. For instance, the one or more OCSAs may comprise one or more salts of a fatty acid(s), such as ricinoleic acid, and/or one or more cucurbituril compounds, such as CB[5], CB[6], CB[7], CB[8], or any mixture thereof, and/or one or more zeolites, and/or one or more halo active aromatic sulfonamide compounds. Additionally or alternatively, the LPTS, in accordance with certain embodiments of the invention, may comprise a TAF, a nonwoven fabric, or a combination thereof. The LPTS may comprise TAF including, for example, a third group of one or more OCSAs dispersed throughout a thickness of the TAF, applied to a top surface of the TAF, and/or applied to a bottom surface of the TAF. For instance, the one or more OCSAs comprise one or more salts of a fatty acid(s), such as ricinoleic acid, and/or one or more cucurbituril compounds, such as CB[5], CB[6], CB[7], CB[8], or any mixture thereof, and/or one or more zeolites, and/or one or more halo active aromatic sulfonamide compounds. In accordance with certain embodiments of the invention, the one or more OCSAs present in or on the backsheet, the second group of one or more OCSAs, and/or the third group of one or more OCSAs may be the same or different from each other. In accordance with certain embodiments of the invention, the absorbent article may further comprise a backsheet-coating, such as the coating described above in relation to the microporous film. The backsheet-coating may be disposed a top surface of the backsheet, wherein the backsheet-coating is located between the backsheet and the absorbent core. Additionally or alternatively, the absorbent article may further comprise an ADL-coating, such as the coating described above in relation to the microporous film, disposed onto a top surface and/or back surface of the of the apertured film. Additionally or alternatively, the absorbent article may further comprise a TAF-coating, such as the coating described above in relation to the microporous film, disposed onto a top surface and/or back surface of the of the TAF.

In accordance with certain embodiments of the invention, the backsheet-coating and/or the ADL-coating and/or the TAF-coating may comprise from about 0.0001 wt. % to about 50 wt. % of the one or more salts of a fatty acid(s), such as ricinoleic acid, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more salts of a fatty acid(s), such as ricinoleic acid, and/or at most about any of the following: 50, 40, 30 and 25 wt. % of the one or more salts of a fatty acid(s), such as ricinoleic acid. Additionally or alternatively, from about 0.0001 wt. % to about 50 wt. % of the one or more halo active aromatic sulfonamide compounds, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more halo active aromatic sulfonamide compounds, and/or at most about any of the following: 50, 40, 30 and 25 wt. % of the one or more halo active aromatic sulfonamide compounds. Additionally or alternatively, the backsheet-coating and/or the ADL-coating and/or the TAF-coating comprises from about 0.0001 wt. % to about 50 wt. % of the one or more cucurbituril compounds, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more cucurbituril compounds, and/or at most about any of the following: 50, 40, 30 and 25 wt. % of the one or more cucurbituril compounds. Additionally or alternatively, the backsheet-coating and/or the ADL-coating and/or the TAF-coating comprises from about 0.0001 wt. % to about 50 wt. % of the one or more zeolites, such as at least about any of the following: 0.0001, 0.001, 0.01, 0.02, 0.05, 0.08, 0.1, 0.5, 0.2, 0.5, 0.8, 1, 2, 5, 8, 10, 15, 20, and 25 wt. % of the one or more zeolites, and/or at most about any of the following: 50, 40, 30 and 25 wt. % of the one or more zeolites. Additionally or alternatively, the backsheet-coating and/or the ADL-coating and/or the TAF-coating has a coating-thickness from about 10 microns to about 2000 microns, such as at least about any of the following: 10, 15, 20, 25, 30, 35, 40, 45, 50, 80, 100, 150, 200, 250, 300, 500, 800, and 1000 microns, and/or at most about any of the following: 2000, 1800, 1500, 1200, and 1000 microns.

In accordance with certain embodiments of the invention, the film comprises a multi-layer film including the breathable film, such as those described and disclosed herein. The multi-layer film may comprise at least a first skin layer and a core layer, wherein the first skin layer has a first thickness and the core layer has a second thickness, and the first thickness is less than the second thickness. In some embodiments, the multi-layer film further comprises a second skin layer, wherein the core layer is located between the first skin layer and the second skin layer. Additionally or alternatively, at least one of the first skin layer and the core layer comprises a breathable film (e.g., microporous film) as described and disclosed herein. Additionally or alternatively, the core layer comprises a monolithic film layer. The monolithic film layer, for example, may comprise at least one highly breathable polymer. The highly breathable polymer(s), in accordance with certain embodiments of the invention, may comprise at least one of a thermoplastic urethane (TPU), a polyether block amide copolymer (e.g., PEBAX® from Arkema Group or Vetsamid® E from Evonik), or a copolyester thermoplastic elastomer (e.g., ARNITEL® from DSM Engineering Plastics, HYTREL® from E.I. DuPont de Nemours and Company). The monolithic film layer, in accordance with certain embodiments of the invention, may comprise a polyether-block-ester copolymer including (i) soft blocks comprising polyethylene glycol and (ii) hard blocks comprising polybutylterephthalate. The monolithic film layer, in accordance with certain embodiments of the invention, may comprises a copolymer of isotactic polypropylene microcrystalline regions and random amorphous regions.

In accordance with certain embodiments of the invention, the film (e.g., single layer film or multi-layer film) is melt-extruded directly onto the absorbent core. As noted above, the film may be a breathable film (e.g., vapor permeable) or a non-breathable film. In this regard, a multilayer film may be a co-extruded multilayer film. Alternatively, the film may be thermally and/or adhesively bonded directly to the absorbent core, such as by a plurality of individual bond sites that directly fuse adjacent portions of the absorbent core to the film.

Alternatively, the film, such as a breathable film or non-breathable film described and disclosed herein, may be indirectly bonded to the absorbent core via an adhesive layer located between the film and the absorbent core. In this regard, an adhesive layer may be located between and adhering the absorbent core to the film. In accordance with certain embodiments of the invention, the adhesive layer may have a basis weight from about 0.2 to about 5 gsm, such as at least about any of the following: 0.2, 0.4, 0.5, 0.6, 0.8, 1, 1.2, 1.4, 1.5, 1.6, 1.8, 2, 2.2, 2.4, and 2.5 gsm, and/or at most about any of the following: 5, 4,5, 4, 3.5, 3, and 2.5 gsm. In accordance with certain embodiments of the invention, the adhesive layer may comprise a discontinuous layer including a plurality of individual adhesive deposition locations. Additionally or alternatively, the adhesive layer comprises one or more continuous streaks, one or more discontinuous streaks, or a combination thereof. For example, the adhesive layer comprises one or more continuous streaks that do not overlap in accordance with certain embodiments. Additionally or alternatively, the adhesive layer may comprise one or more continuous streaks, in which a first plurality of continuous streaks define intersection regions defined by overlapping portions of the first plurality of continuous streaks. Alternatively, the adhesive layer comprises a continuous layer.

The absorbent article, in accordance with certain embodiments of the invention, comprises a diaper, such as an adult diaper or a baby diaper, or a feminine care product, such as a pad or a liner, or a pet pad, such as a puppy pad.

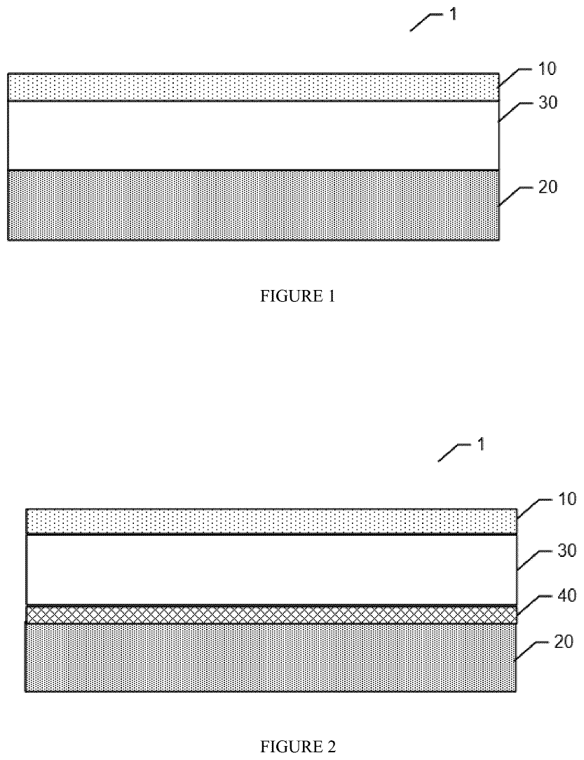

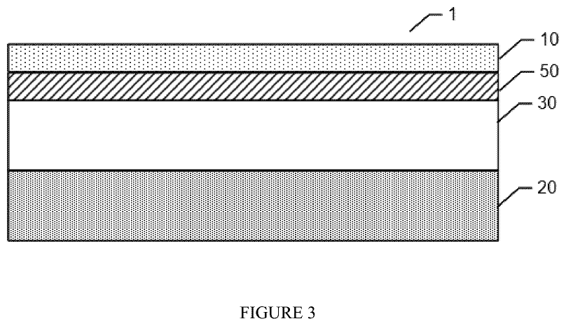

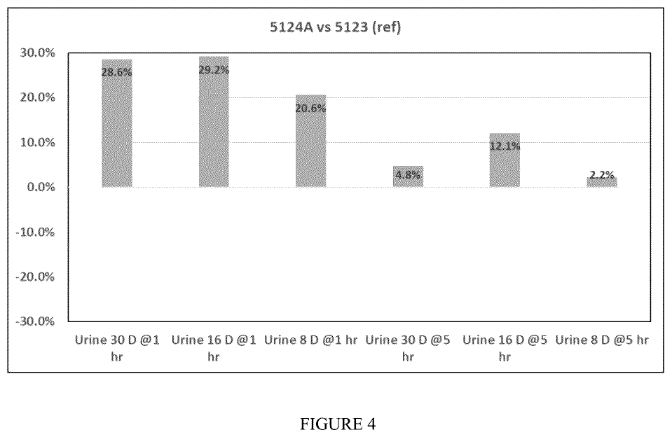

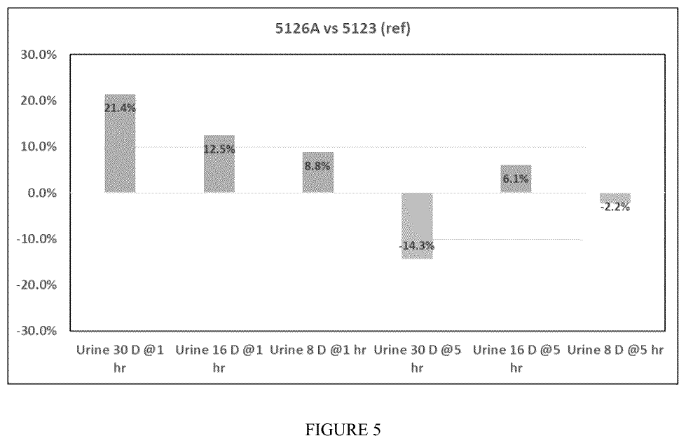

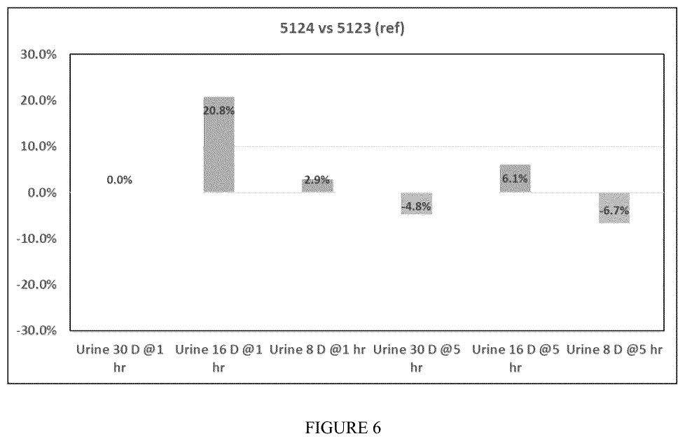

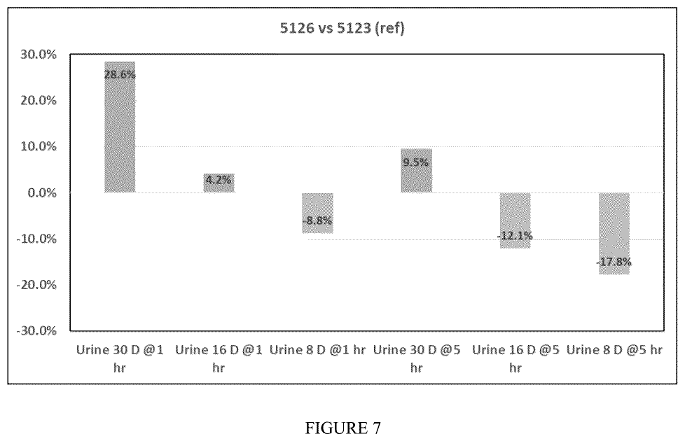

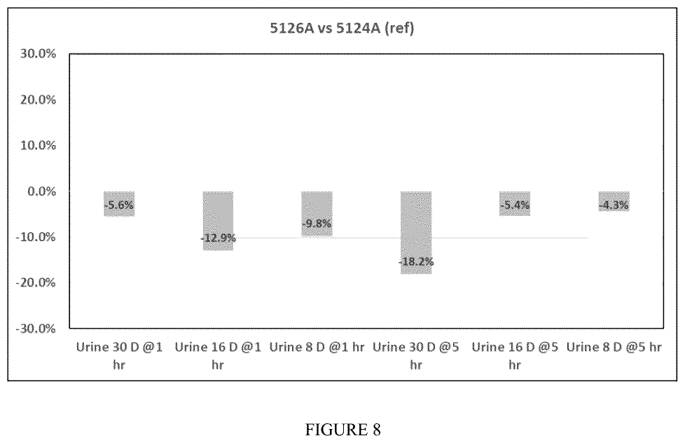

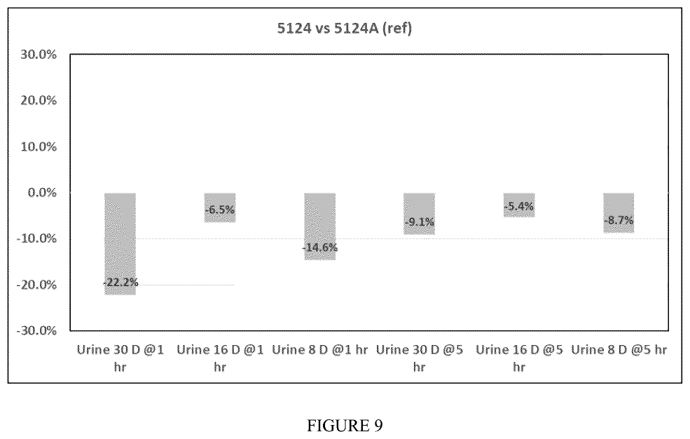

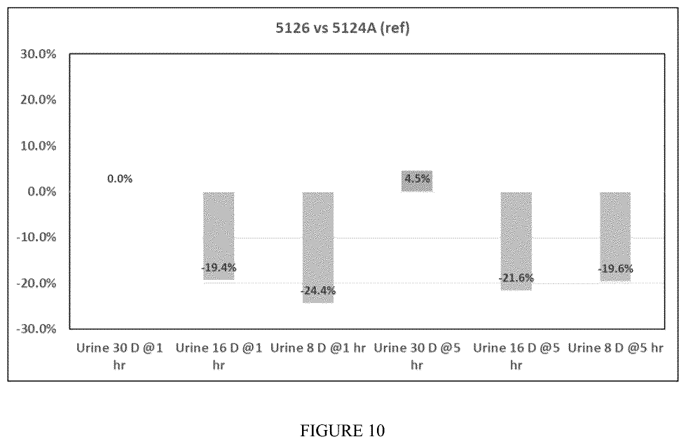

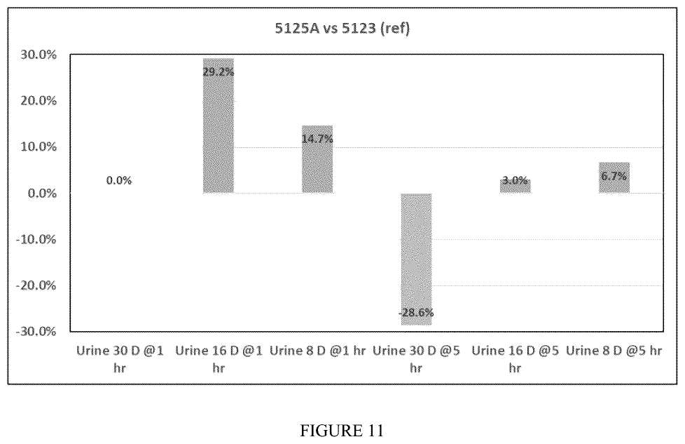

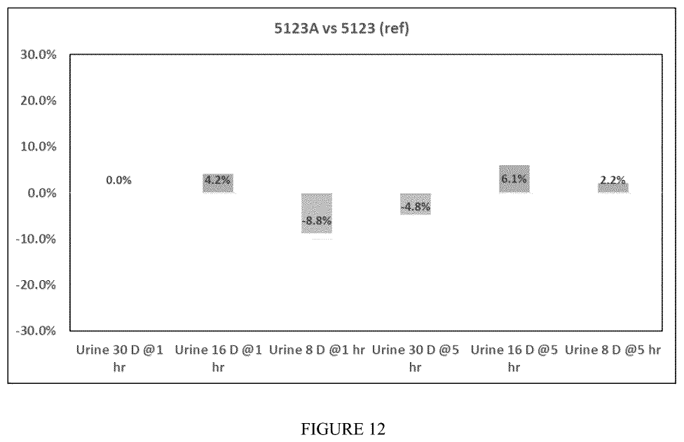

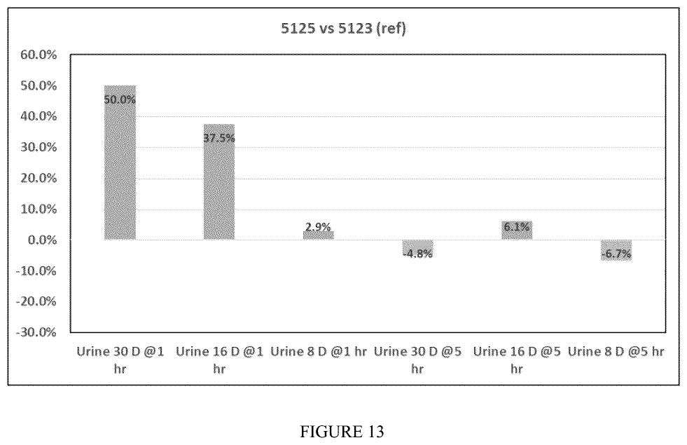

FIG. 1, for example, illustrates an absorbent article 1 including a nonwoven topsheet 10 that may be positioned closest to a user's skin and a backsheet 20, such as those described and disclosed herein, that may be located further away from the user's skin during use. The absorbent article 1 also includes an absorbent core 30 located between the topsheet 10 and backsheet 20. FIG. 2 illustrates an absorbent article 1 including a nonwoven topsheet 10 that may be positioned closest to a user's skin and a backsheet 20, such as those described and disclosed herein, that may be located further away from the user's skin during use. The absorbent article 1 includes an absorbent core 30 and an adhesive layer 40, in which the backsheet is indirectly bonded to the absorbent core via the adhesive layer. FIG. 3 illustrates an absorbent article 1 including an acquisition distribution layer 50 located between a topsheet 10 and an absorbent core 30 in accordance with certain embodiments of the invention. The absorbent article 1 also includes a backsheet 20 bonded to the absorbent core 30, such that the absorbent core is located between the acquisition distribution layer and the backsheet. In this regard, the acquisition distribution layer may comprise a nonwoven web or fabric formed from spunbond fibers, meltblown fibers, staple fibers, cellulosic fibers (e.g., natural and/or synthetic cellulose fibers) and/or an apertured film as referenced herein. In accordance with certain embodiments of the invention, the acquisition distribution layer comprises a high-loft nonwoven web or fabric and/or an apertured film. The acquisition distribution layer, for example, may receive fluids from the topsheet and distribute the fluid over a greater area and transports the fluid to the absorbent core over the greater area. As noted above, the acquisition distribution layer may comprise a high-loft material to prevent rewet (e.g., liquid traveling out of the absorbent article back to the user).

In yet another aspect, the present invention provides a method of making an absorbent article comprising the following: (i) providing or forming a liquid permeable topsheet (LPTS), such as a nonwoven fabric and/or a TAF; (ii) providing or forming a backsheet comprising a film, such as a breathable film or non-breathable film as described and disclosed herein; (iii) providing or forming an absorbent core, wherein the absorbent core is located directly or indirectly between the LPTS and the backsheet; and (iv) bonding the backsheet directly or indirectly to the absorbent core. In accordance with certain embodiments of the invention, the step of directly or indirectly bonding the backsheet to the absorbent core comprises directly bonding the backsheet to the absorbent core via the formation of one or more thermal bonds between the backsheet to the absorbent core, via melt-extruding a precursor film directly onto the absorbent core either before or after incrementally stretching the precursor film to form a microporous film, or via melt-extruding the film directly onto the absorbent core. Alternatively, the step of directly or indirectly bonding the backsheet to the absorbent core comprises indirectly bonding the backsheet to the absorbent core via addition of an adhesive layer between the backsheet to the absorbent core.

In accordance with certain embodiments of the invention, the method may comprise providing or forming an ADL and disposing the ADL directly or indirectly between the LPTS and the absorbent core. As noted above, the ADL may comprise an apertured-film, a high-loft nonwoven fabric, or a combination thereof. In accordance with certain embodiments of the invention, the ADL comprises an apertured-film that includes a second group of one or more OCSAs dispersed throughout a thickness of the apertured-film, applied to a top surface of the apertured film, and/or applied to a bottom surface of the apertured-film. Additionally or alternatively, the LPTS, in accordance with certain embodiments of the invention, may comprise a nonwoven fabric and/or TAF. The LPTS may comprise TAF including, for example, a third group of one or more OCSAs dispersed throughout a thickness of the TAF, applied to a top surface of the TAF, and/or applied to a bottom surface of the TAF. For instance, the one or more OCSAs may comprise one or more salts of a fatty acid(s), such as ricinoleic acid, and/or one or more cucurbituril compounds, such as CB[5], CB[6], CB[7], CB[8], or any mixture thereof, and/or one or more halo active aromatic sulfonamide compounds of Formula (I). In accordance with certain embodiments of the invention, the one or more OCSAs present in or on the backsheet, the second group of one or more OCSAs, and/or the third group of one or more OCSAs may be the same or different from each other.

### Examples

The present disclosure is further illustrated by then following examples, which in no way should be construed as being limiting. That is, the specific features described in the following examples are merely illustrative and not limiting.

### Listing of Materials

Heritage Plastics CF7030PE: is a master batch including calcium carbonate (i.e., CaCO₃) therein.

Westlake EC474AA (WL 474): is a low density polyethylene resin.

ExxonMobil Exceedrm 3518PA (EM 3518): is an linear low density polyethylene resin made with ethylene 1-hexene copolymer.

Chevron Phillips Marlex® HMN 6060 (CPC 6060): is a high density polyethylene resin made with ethylene hexene copolymer.

AMPACET 1001102-N: is an odor control master batch designed to absorb nitrogen and sulfur containing odor causing molecules.

### Example Set

A variety of sample films were formulated for testing and comparison, such as for odor sequestering. Table 1 provides a summary for each sample film formed and tested (i.e., Examples 1-8).

TABLE 1

| | | | | Example 3 | | | | | |
|---|---|---|---|---|---|---|---|---|---|
| | | | | Film made | | | | Example 7 | Example 8 |
| | | | Example 2 | with resins | Example 4 | | | Film made | Film made |
| | | Example 1 | Film made | and Odor | Film made | Example 5 | Example 6 | with resins, | with resins, |
| | | Film made | with resins | control MB | with resins | Film made | Film made | CaCO3 and | CaCO3 and |
| | | with resins | only and is | and is | and Oder | with resins | with resins | Oder | Odor |
| | | only | activated | activated | control MB | and CaCO3, | and CaCO3 | control MB | control MB |
| | | Non- | Non- | Non- | Non- | and is | Non- | and is | Non- |
| | | breathable | breathable | breathable | breathable | activated | breathable | activated | breathable |
| | | non-filled | non-filled | non-filled | non-filled | Breathable | filled | Breathable | filled |
| | Description | film | film | film | film | films | film | film | film |
| IDs | Film Codes | M16-5123 | M16-5123A | M16-5124 | M16-5124A | M16-5125 | M16-5125A | M16-5126 | M16-5126A |
| Formulation | CF7030PE | | | | | 65 | 65\| | 65 | 65 |
| | WL 474 | 5 | 5 | 5 | 5 | 5 | 5 | 5 | 5 |
| | EM 3518 | 75 | 75 | 65 | 65 | 15 | 15 | 5 | 5 |
| | CPC 6060 | 20 | 20 | 20 | 20 | 15 | 15 | 15 | 15 |
| | Ampacet | | | 10 | 10 | | | 10 | 10 |
| | 1001102-N | | | | | | | | |
| | Total | 100.0 | 100.0 | 100.0 | 100.0 | 100.0 | 100.0 | 100.0 | 100.0 |
| Properties | BW (gsm) | 20.4 | 20.9 | 24.6 | 18.1 | 17.3 | 18.2 | 19.2 | 19.5 |
| | Mocon | — | — | — | — | 3554 | — | 4387 | — |

Although summarized in Table 1 above, Example 1 (Film Code M16-5123) is a base film that is devoid of the odor control master batch and filler (i.e., calcium carbonate).

Example 2 (Film Code M16-5123A) was formed from the same composition as Example 1. Example 2, however, was subjected to an activation step (e.g., incremental stretching). Similar to Example 1, Example 2 is also a non-breathable film.

Example 3 (Film Code M16-5124) included the odor control master batch. This film was devoid of filler, and was subjecting to an activation step (e.g., incremental stretching). Example 3 was a non-breathable film.

Example 4 (Film Code M16-5124A) was formed from the same composition as Example 3, but was not subjected to an activation step (e.g., incremental stretching). This film was non-breathable.

Example 5 (Film Code M16-5125) was a breathable film that included the calcium carbonate-containing master batch. This film was devoid of the odor control master batch, and subjected to an activation step (e.g., incremental stretching).

Example 6 (Film Code M16-5125A) was formed from an identical composition as Example 5, but was not subjected to an activation step (e.g., incremental stretching). Example 6 was a non-breathable film.

Example 7 (Film Code M16-5126) was a breathable film that included both the odor control master batch and the calcium carbonate-containing master batch. This film was also subjected to an activation step (e.g., incremental stretching).

Example 8 (Film Code M16-5126A) was formed from an identical composition as Example 7, but this film was not subjected to an activation step (e.g., incremental stretching). This film was non-breathable.

### Testing Protocol:

Headspace samples prepared in 10-L Tedlar air sample bags.

Insult of 1 mL real human urine in weigh pan.

Film material draped over tented stainless steel screen material.

Samples held in 98° F. oven during holding time of 1 to 5 hours.

Separate samples prepared for 1 hour and 5 hour time points.

Headspace presented through olfactometer at multiple dilution levels.

### Study I: Objective—Investigate the Impact of Odor Control Master Batch, CaCO₃, and Activation on Urine Odor Reduction.

In this study, four films—5124A, 5126A, 5124 and 5126—were individually compared against the 5123 film, which was the reference.

The sole addition of odor control master batch did not result in urine odor reduction during both the 1-hour and 5-hour testing intervals as illustrated in FIG. 30. This could potentially be attributed to the resin coating on the odor actives, which may have negatively influenced its efficacy (5124A film vs. 5123 film).

When odor control master batch was combined with CaCO₃, there were indications of odor reduction after 1 hour as illustrated in FIG. 31. By the 5-hour mark, a noticeable reduction in odor was observed (5126A film vs. 5123 film). This phenomenon could be due to the pores in CaCO₃ adsorbing the odor molecules, however, we do not wish to be bound by this theory. Similar trends were observed with the addition of odor control master batch and activation (5124 film vs. 5123 film) as illustrated by FIG. 32. Although not wishing to be bound by the following theory, but it is believed that the activation process comprising the stretching of the film aids in the exposure of the odor actives embedded within.

The inclusion of CaCO₃ and utilization of activation resulted in odor reduction at both 1 hour and 5 hours, particularly at lower dilution levels (5126 film vs. 5123 film) as illustrated by FIG. 33.

Similar observations were made when three films—5126A, 5124 and 5126—were individually compared against the film containing the odor control MB (5124A) as illustrated in FIGS. 34, 35, and 36, respectively.

Study II: Objective—to Evaluate the Necessity of Odor Control Master Batch in Conjunction with CaCO₃ and Activation for Effective Odor Reduction.

In this study, three films—5125A, 5123A and 5125—were individually compared against the 5123 film.

In certain instances, where CaCO₃ and activation successfully mitigated minor urine odors, in which potential factors discussed in the previous section may contribute to this effect, as illustrated in FIGS. 37 and 38. However, for significant odor mitigation, the inclusion of odor control master batch appears to be imperative as illustrated in FIG. 39. Utilizing all three components—odor control master batch, CaCO₃, and activation—demonstrated a synergistic effect, yielding the most effective odor reduction performance.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A breathable film, comprising: a vapor-permeable and liquid impermeable (VPLI) film comprising a microporous film comprising a plurality of micropores, wherein the microporous film includes (a) a first outermost surface, (b) a second outermost surface, (c) a thickness extending between the first outermost surface and the second outermost surface, and (d) at least one odorous compound sequestering agent (OCSA) dispersed throughout the thickness of the film, wherein the at least one OCSA comprises (i) one or more salts of ricinoleic acid, and/or (ii) one or more cucurbituril compounds and/or (iii) one or more zeolites, and/or (iv) one or more halo active aromatic sulfonamide compounds.

2. The breathable film of claim 1, wherein the one or more salts of ricinoleic acid comprises a transition metal and/or a post-transition metal.

3. The breathable film of claim 1, wherein the one or more salts of ricinoleic acid comprises zinc ricinoleate.

4. The breathable film of claim 1, wherein the one or more salts of ricinoleic acid comprises from about 0.0001 wt. % to about 40 wt. % of the microporous film.

5. The breathable film of claim 1, wherein the microporous film comprises a single-layer film.

6. The breathable film of claim 1, wherein the microporous film comprises a multi-layer film comprising from 2 to about 10 individual microporous film layers bonded together.

7. The breathable film of claim 1, wherein the thickness of the microporous film comprises from about 10 to about 500 microns.

8. The breathable film of claim 1, wherein the microporous film comprises a polymer component and an additive component, wherein the additive component comprises (i) one or more salts of ricinoleic acid, and/or one or more cucurbituril compounds and/or one or more zeolites, and/or one or more halo active aromatic sulfonamide compounds, and (ii) a pore-forming filler material comprising a plurality of filler-particles.

9. The breathable film of claim 1, wherein the polymer component of the microporous film comprises a polyolefin, such as a polyethylene or copolymer thereof or a polypropylene or a copolymer thereof or a blend of a first polyolefin and a second polyolefin.

10. The breathable film of claim 1, wherein one or more cucurbituril compounds are present in an uncomplexed form.

11. The breathable film of claim 1, wherein one or more cucurbituril compounds are selected from CB[5], CB[6], CB[7], CB[8], or any mixture thereof.

12. The breathable film of claim 1, wherein the microporous film has a MVTR from 200 to 20,000 g/m²/24 hours as determined according to WSP 70.4(08).

13. The breathable film of claim 1, further comprising a coating adjacent the first outermost surface, the second outermost surface, or both; wherein the coating comprising at least one OCSA comprising one or more salts of ricinoleic acid dispersed throughout the coating and/or one or more halo active aromatic sulfonamide compounds dispersed throughout the coating and/or one or more cucurbituril compounds dispersed throughout the coating.

14. A method of forming a breathable film, comprising:
(i) forming a polymer melt;
(ii) adding a pore-forming filler material to the polymer melt;
(iii) adding a dry masterbatch to the polymer melt, wherein the dry masterbatch comprises at least one odorous compound sequestering agent (OCSA) comprising (a) one or more salts of ricinoleic acid, and/or (b) one or more cucurbituril compounds, and/or (c) one or more zeolites, and/or (d) one or more halo active aromatic sulfonamide compounds;
(iv) admixing the pore-forming filler material and the dry masterbatch into the polymer melt;
(v) melt extruding the polymer melt including the pore-forming filler material and at least one OCSA to form an intermediate film;
(vi) incrementally stretching the intermediate film in a machine-direction and/or a cross-direction to form the breathable film.

15. The method of claim 14, wherein the dry masterbatch includes a polymer matrix component and the at least one OCSA is dispersed throughout the polymer matrix.

16. The method of claim 15, wherein the polymer matrix comprises a matrix polymer corresponding to a polymer component of the polymer melt.

17. An absorbent article, comprising:
(i) a liquid permeable topsheet (LPTS);
(ii) a backsheet comprising a breathable film according to claim 1;
(iii) an absorbent core, wherein the absorbent core is located directly or indirectly between the LPTS and the backsheet.

18. The absorbent article of claim 17, further comprising an acquisition distribution layer (ADL) located directly or indirectly between the LPTS and the absorbent core.

19. A method of making an absorbent article, comprising:
(i) providing or forming a liquid permeable topsheet (LPTS);
(ii) providing or forming a backsheet comprising a breathable film according to claim 1;
(iii) providing or forming an absorbent core, wherein the absorbent core is located directly or indirectly between the LPTS and the backsheet; and
(iv) bonding the backsheet directly or indirectly to the absorbent core.

20. The method of claim 19, wherein the step of directly or indirectly bonding the backsheet to the absorbent core comprises directly bonding the backsheet to the absorbent core via the formation of one or more thermal bonds between the backsheet to the absorbent core, via melt-extruding a precursor film directly onto the absorbent core either before or after incrementally stretching the precursor film to form a microporous film, or via melt-extruding the film directly onto the absorbent core.
